(19) United States
(12) Patent Application Publication
(10) Pub. No.: US 2003/0114512 A1
(43) Pub. Date: Jun. 19, 2003

(54) Compounds with 5-ht2 and 5-ht1a agonist activity for treating glaucoma

(76) Inventors: Robert J Collier JR., Arlington, TX (US); Thomas R Dean, Weatherford, TX (US); Mark R Hellberg, Highland Village, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/221,116

(22) PCT Filed: Feb. 20, 2001
(86) PCT No.: PCT/US01/05432

(51) Int. Cl. A61K 31/405; A61K 31/20; A61K 31/557

(52) U.S. Cl. 514/419; 514/560; 514/573

(57) ABSTRACT

Compounds with both 5-HT₂ and 5-HT_{1A} agonist activity which are useful for lowering and controlling IOP and the treatment of glaucomatous optic neuropathy.

The present invention is directed to compounds with 5-HT₂ and 5-HT_{1A} agonist activity useful for lowering and controlling intraocular pressure (IOP) and the treatment of glaucomatous optic neuropathy.

### BACKGROUND OF THE INVENTION

Glaucoma is a family of diseases, each of which is distinguished by a particular characteristic of that disease form. Primary open-angle glaucoma (POAG) is characterized by typical glaucomatous changes to optic nerve head topography, arcurate scotomas in the visual field, an open-angle, and is usually associated with elevated intraocular pressure. Normotension glaucoma (NTG) or low tension glaucoma is very similar to POAG except the IOP for these patients is in the normal range. Other forms of glaucoma include closed-angle glaucoma and pigmentary dispersion glaucoma. All these forms of glaucoma are similar in that patients suffer from the continued loss of nerve fiber layer and vision. Current therapies for the treatment of glaucoma, in particular POAG and NTG, strive to slow the progression of the disease by lowering and controlling intraocular pressure. This is done either by IOP lowering drugs or in more difficult cases by argon laser trabeculoplasty (ALT) and/or by glaucoma filtration surgery (GFS). Long-term studies of the effects of lowering IOP (even in NTG patients) have been shown to be effective in slowing the disease progression in some patients. Unfortunately, there are patients who continue to progress despite having their IOP lowered.

To address these patients, drug therapies that both lower IOP and provide additional protection to the retina and optic nerve head have been developed. Compounds such as betaxolol and brimonidine have been shown to be neuroprotective in animal models. Both have been suggested to provide neuroprotection in glaucoma by direct penetration to the back of the eye after topical ocular administration. Betaxolol's neuroprotection properties are believed to arise from its calcium channel blocking activities and its ability to stimulate the expression of key neuroprotective factors such as CNTF, bFGF, and BDNF. Brimonidine is an α₂ agonist and is believed to stimulate the production of bFGF.

Serotonergic—agonists have been reported as being neuroprotective in animal models and many of these agents have been evaluated for the treatment of acute stroke among other indications. This class of compounds has been disclosed for the treatment of glaucoma (lowering and controlling IOP), see e.g., WO 98/18458 (DeSantis, et al) and EP 0771563A2 (Mano, et al.). Osborne, et al. (Ophthalmologica, Vol. 210:308-314, 1996) teach that 8-hydroxydipropylaminotetralin (8-OH-DPAT) (a 5-HT_{1A} agonist) reduces IOP in rabbits. Wang, et al. (Current Eye Research, Vol. 16(8):769-775, August 1997, and IVOS, Vol. 39(4):2236, March, 1998) disclose that 5-methylurapidil, an α_{1A} antagonist and 5-HT_{1A} agonist lowers IOP in the monkey, but due to its α_{1A} receptor activity. Also, 5-HT_{1A} antagonists are disclosed as being useful for the treatment of glaucoma (elevated IOP) (e.g. WO 92/0338, McLees). Furthermore, DeSai, et al. (WO 97/35579) and Macor, et al. (U.S. Pat. No. 5,578,612) disclose the use of 5-HT₁ and 5-HT₁₋ₗᵢₖₑ agonists for the treatment of glaucoma (elevated IOP). These anti-migraine compounds are 5-HT_{1B,D,E,F} agonists, e.g., sumatriptan and naratriptan and related compounds.

In a co-pending application, (PCT US99/19888), 5-HT₂ agonists have been shown to be a new class of potent ocular hypotensive agents useful for the treatment of elevated IOP and glaucoma.

### SUMMARY OF THE INVENTION

This invention is directed to compounds that have potent agonist activity at the 5-HT₂ and 5-HT_{1A} receptors. They are useful for lowering and controlling IOP and treating glaucomatous optic neuropathy (Compounds). The Compounds are delivered topically and are believed to be able to penetrate directly to the back of the eye and protect retinal ganglion cells and cells associated with the optic nerve head and lamina cribrosa.

### DESCRIPTION OF PREFERRED EMBODIMENTS

Surprisingly, Compounds of the present invention have both 5-HT₂ and 5-HT_{1A} activity and are useful for lowering and controlling IOP and treating glaucomatous optic neuropathy via topical ocular administration. The IOP lowering effects of the Compounds are attributable to their 5-HT₂ activity. Neuroprotection is derived from their 5-HT_{1A} activity. Compounds within this invention are characterized by the following parameters which can be determined using the below described methods. Compounds of this invention have ligand binding IC₅₀ values against 5-HT₂ receptors ranging up to about 100 nM (preferably less than 50 nM). Compounds of this invention are either full or partial agonists with EC₅₀ values ranging up to about 1 μM (preferably less than 500 nM). These Compounds also have potent affinity for 5-HT_{1A} receptors with IC₅₀ values that range up to about 500 nM (preferably less than 100 nM). These Compounds are also either full or partial agonists with IC₅₀ values ranging up to about 1 μM (preferably less than 500 nM).

Compounds of the present invention are exemplified by α-Methyl 5-hydroxytryptamine (3-(2-aminopropyl)-1H-indol-5-ol) which is a potent 5-HT₂ agonist based on ligand binding and functional assays (Table 1). This Compound also has potent affinity for the 5-HT_{1A} receptor (Table 2 and J. Med. Chem. 33, 755 (1990)) and is an agonist. Topical ocular delivery to hypertensive monkey eyes results in a potent reduction in IOP (Table 3). It is also neuroprotective in the rat photooxidative induced retinopathy model after ip administration (Table 4). α-Methyl 5-hydroxytryptamine is believed to penetrate to the back of the eye in high enough concentrations to confer neuroprotection upon topical ocular dosing.

α-Methyl 5-hydroxytryptamine and other Compounds representative of the present invention are shown below.

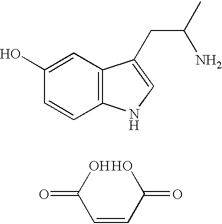

### α-Methyl 5-hydroxytryptamine maleate (Compound 1)

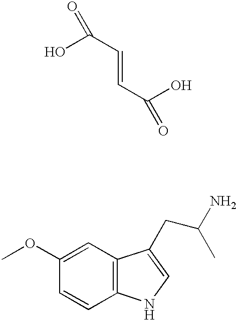

### α-Methyl 5-methoxytryptamine fumarate (Compound 2)

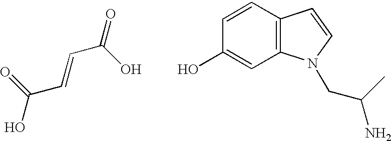

### 1-(2-aminopropyl) 6-hydroxyindole fumarate (Compound 3)

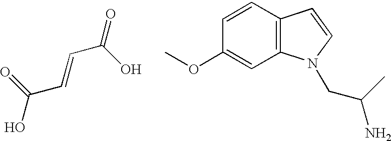

### 1-(2-aminopropyl) 6-methoxyindole fumarate (Compound 4)

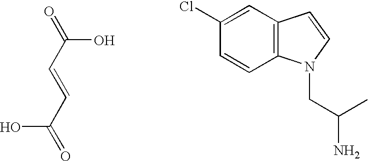

### 1-(2-aminopropyl) 5-chloroindole fumarate (Compound 5)

These Compounds can be made by methods readily available to those skilled in the art. Receptor binding and agonist activity according to this invention can be determined using the following methods.

### METHOD 1

### 5-HT₂ Receptor Binding Assay

To determine the affinities of serotonergic compounds at the 5-HT₂ receptors, their ability to compete for the binding of the agonist radioligand [¹²⁵I]DOI to brain 5-HT₂ receptors is determined as described below with minor modification of the literature procedure [Neuropharmacology, 26, 1803 (1987)]. Aliquots of post mortem rat or human cerebral cortex homogenates (400 μl) dispersed in 50 mM TrisHCl buffer (pH 7.4) are incubated with [¹²⁵I]DOI (80 pM final) in the absence or presence of methiothepin (10 μM final) to define total and non-specific binding, respectively, in a total volume of 0.5 ml. The assay mixture is incubated for 1 hour at 23° C. in polypropylene tubes and the assays terminated by rapid vacuum filtration over Whatman GF/B glass fiber filters previously soaked in 0.3% polyethyleneimine using ice-cold buffer. Test compounds (at different concentrations) are substituted for methiothepin. Filter-bound radioactivity is determined by scintillation spectrometry on a beta counter. The data are analyzed using a non-linear, iterative curve-fitting computer program [Trends Pharmacol. Sci., 16, 413 (1995)] to determine the compound affinity parameter. The concentration of the compound needed to inhibit the [¹²⁵I]DOI binding by 50% of the maximum is termed the IC₅₀ or Kᵢ value.

### METHOD 2

### 5-HT₂ Functional Assay: Phosphoinositide (PI) Turnover Assay

The relative agonist activity of serotonergic compounds at the 5-HT₂ receptor can be determined in vitro using the ability of the compounds to stimulate the production of [³H]inositol phosphates in [³H]myo-inositol-labeled A7r5 rat vascular smooth muscle cells by their ability to activate the enzyme phospholipase C. These cells are grown in culture plates, maintained in a humidified atmosphere of 5% CO₂ and 95% air and fed semi-weekly with Dulbecco's modified Eagle medium (DMEM) containing 4.5 g/l glucose and supplemented with 2 mM glutamine, 10 μg/ml gentamicin, and 10% fetal bovine serum. For the purpose of conducting the phosphoinositide (PI) turnover experiments, the A7r5 cells are cultured in 24-well plates as previously [J. Pharmacol. Expt. Ther., 286, 411 (1998)]. Confluent cells are exposed for 24-30 hrs to 1.5 μCi [³H]-myo-inositol (18.3 Ci/mmol) in 0.5 ml of serum-free medium. Cells are then rinsed once with DMEM/F-12 containing 10 mM LiCl prior to incubation with the test agent (or solvent as the control) in 1.0 ml of the same medium for 1 hr at 37° C., after which the medium is aspirated and 1 ml of cold 0.1 M formic acid added to stop the reaction. The chromatographic separation of [³H]-inositol phosphates ([³H]-IPs) on an AG-1-X8 column is performed as previously described [J. Pharmacol. Expt. Ther. 286, 411 (1998)] with sequential washes with H₂O and 50 mM ammonium formate, followed by elution of the total [³H]-IPs fraction with 1.2 M ammonium formate containing 0.1 M formic acid. The eluate (4 ml) is collected, mixed with 15 ml scintillation fluid, and the total [³H]-IPs determined by scintillation counting on a beta counter. Concentration-response data are analyzed by the sigmoidal fit function of the Origin Scientific Graphics software (Microcal Software, Northampton, Mass.) to determine agonist potency (EC₅₀ value) and efficacy (Emax). Serotonin (5-HT) is used as a positive control (standard) agonist compound and the efficacy of test compounds is compared to that of 5-HT (set at 100%). The concentration of the compound needed to stimulate the production of [³H]-IPs by 50% of the maximum response is termed the EC₅₀ value.

The above procedures were used to generate the data shown in Table 1.

TABLE 1
5-HT₂ Receptor Binding and Functional Data.

| | IC₅₀, nM | EC₅₀, nM | Efficacy |
|---|---|---|---|
| Compound | (SEM) | (SEM) | (Emax, %) |
| α-Methyl 5-hydroxytryptamine | 3.5 | 189 | 104 |
| maleate | | | |
| α-Methyl 5-methoxytryptamine | 4.35 | 206 | 81 |
| fumarate | | | |
| 1-(2-aminopropyl) 6-hydroxyindole | 4.9 | 577 | 86 |
| fumarate | | | |
| 1-(2-aminopropyl) 6- | 2.5 | 255 | 88 |
| methoxyindole fumarate | | | |
| 1-(2-aminopropyl) 5-chloroindole | 37.1 | 1390 | 104 |
| fumarate | | | |

### METHOD 3

### 5-HT_{1A} Receptor Binding Assay

5-HT_{1A} binding studies were performed with human cloned receptors expressed in Chinese hamster ovary (CHO) cells using (³H)8-OH DPAT as the ligand. Membranes from Chinese hamster ovary cells (CHO) expressing cloned 5-HT_{1A} receptors (manufactured for NEN by Biosignal, Inc., Montreal, Canada) were homogenized in approximately 40 volumes of 50 mM Tris pH 7.4 for 5 sec. Drug dilutions were made using a Beckman Biomek 2000 robot (Beckman Instruments, Fullerton, Calif.). Incubations were conducted with membrane prep, test compounds, and 0.25 nM [³H]8-OH-DPAT (NEN, Boston, Mass.) in the same buffer at 27° C. for 1 h. Assays were terminated by rapid vacuum filtration over Whatman GF/B glass fiber filters pre-soaked in 0.3% polyethyleneimine. Bound radioactivity was measured using liquid scintillation spectrometry. Data were analyzed using non-linear curve fitting programs (Sharif et al., J. Pharmac. Pharmacol. 51: 685-694, 1999).

Ligand binding studies can also be run using membrane preparations from calf and rat brain (local source) and human cortex membranes. Specific brain regions were dissected out, homogenized in 10 volumes of 0.32 M sucrose and centrifuged for 10 min at 700×g. The resulting supernatant was centrifuged at 43,500×g for 10 min and the pellet re-suspended in 50 mM Tris-HCl (pH 7.7, 25° C.) using a 10 sec polytron treatment. Aliquots were stored at −140° C. To remove endogenous serotonin, the preps were incubated at 37° C. for 10 min prior to the experiment. Assay incubations were terminated by rapid filtration over Whatman GF/C filters using a Brandel cell harvester. Kᵢ values were calculated using the Cheng-Prusoff equation (De Vry et al., J. Pharm. Exper. Ther. 284:1082-1094, 1998.)

### METHOD 4

### 5-HT_{1A} Functional Assays

The function of Compounds of the present invention can be determined using a variety of methods to assess the functional activity of 5-HT_{1A} agonists. One such assay is performed using hippocampal slices from male Sprague-Dawley rats, measuring the inhibition of forskolin-stimated adenylate cyclase [J. Med. Chem. 42. 36 (1999), J. Neurochem. 56, 1114 (1991), J. Pharm. Exper. Ther. 284:1082 (1998). Rat hippocampal membranes were homogenized in 25 volumes of 0.3 M sucrose containing 1 mM EGTA, 5 mM EDTA, 5 mM dithiothreitol, and 20 mM Tris-HCl, pH 7.4 at 25° C. The homogenate was centrifuged for 10 m in at 1,000×g. The supernatant subsequently was centrifuged at 39,000×g for 10 min. The resulting pellet was re-suspended in homogenization buffer at a protein concentration of approximately 1 mg/ml and aliquots were stored at −140° C. Prior to use, the membranes were rehomogenized in a Potter-Elvehjem homogenizer. Fifty μl of the membrane suspension (50 μg protein) were added to an incubation buffer containing 100 mM NaCl, 2 mM magnesium acetate, 0.2 mM ATP, 1 mM cAMP, 0.01 mM GTP, 0.01 mM forskolin, 80 mM Tris-HCl, 5 mM creatine phosphate, 0.8 U/μl creatine phosphokinase, 0.1 mM IBMX, 1-2 μCi α-[³²P]ATP. Incubations with test compounds (10 min at 30° C.) were initiated by the addition of the membrane solution to the incubation mixture (prewarmed 5 min at 30° C.). [³²P]cAMP was measured according to the method of Salomon (Adv. Cyclic Nucleotide Res. 10:35-55, 1979). Protein was measure using the Bradford (Anal. Biochem 72:248-254, 1976) assay.

Functional activity can also be determined in recombinant human receptors according to the method of Schoeffter et al., (Neuropharm. 36:429-437, 1997). HeLa cells transfected with recombinant human 5-HT_{1A} receptors were grown to confluence in 24-well plates. The cells were rinsed with 1 ml of Hepes-buffered saline (in mM) NaCl 130, KCl 5.4, CaCl₂, 1.8, MgSO₄ 0.8, NaH₂PO₄ 0.9, glucose 25, Hepes 20, pH 7.4, and phenol red 5 mg/l. The cells were labelled with 6 μCi/ml of [³H] adenine (23 Ci/mmol, Amersham, Rahn AG, Zurich, Switzerland) in 0.5 ml of saline at 37° C. for 2 hr. The plates were subsequently rinsed twice with 1 ml of buffered saline containing 1 mM isobutylmethylxanthine. The cells were incubated for 15 min in 1 ml of this solution (37° C.) in the presence or absence of 10 μM forskolin and the test compound. The buffer was then removed and 1 ml of 5% trichloroacetic acid (TCA) containing 0.1 mM cAMP and 0.1 mM ATP was added to extract the samples. After 30 min at 4° C., the TCA extracts were subjected to chromatographic separation on Dowex AG 50W-X4 and alumina columns (Salomon, Methods in Enzymology 195: 22-28, 1991). Cyclic AMP production was calculated as the ratio [³H]cAMP/([³H]cAMP+[³H]ATP).

TABLE 2
5-HT_{1A} Receptor Binding and Functional Data.

| | IC₅₀, nM | EC₅₀, nM | Efficacy |
|---|---|---|---|
| Compound | (SEM) | (SEM) | (Emax, %) |
| α-Methyl 5-hydroxytryptamine | 22.5 | 378 | 100 |
| maleate | | | |
| α-Methyl 5-methoxytryptamine | 26.5 | 570 | 92 |
| fumarate | | | |
| 1-(2-aminopropyl) 6- | 111 | 780 | 89 |
| hydroxyindole fumarate | | | |
| 1-(2-aminopropyl) 6- | 225 | nd | nd |
| methoxyindole fumarate | | | |
| 1-(2-aminopropyl) 5- | 417 | nd | nd |
| chloroindole fumarate | | | |

### METHOD 5

### IOP Response in Lasered (Hypertensive) Eyes of Conscious Cynomolgus Monkeys

Intraocular pressure (IOP) was determined with an Alcon Pneumatonometer after light corneal anesthesia with 0.1% proparacaine. Eyes were washed with saline after each measurement. After a baseline IOP measurement, test compound was instilled in one 30 μL aliquot to the right eyes only of nine cynomolgus monkeys. Vehicle was instilled in the right eyes of six additional animals. Subsequent IOP measurements were taken at 1, 3, and 6 hours. The profile of the IOP response following topical administration is provided in Table 3.

TABLE 3
IOP Response for Representative Compound

| | | | Percent IOP | | |
|---|---|---|---|---|---|
| | | Baseline | Reduction ± SEM | | |
| | Dose, | IOP | Hours after dose | | |
| Example | μg | (mmHg) | 1 | 3 | 6 |
| α-Methyl 5- | 250 | 41.8 | 14.2 | 25.8 | 30.8 |
| hydroxytryptamine maleate | | | | | |
| α-Methyl 5- | 300 | 36.7 | 14 | 23.5 | 22.6 |
| methoxytryptamine | | | | | |
| fumarate | | | | | |
| 1-(2-aminopropyl) 6- | 300 | 36.5 | 19.1 | 30.1 | 27 |
| hydroxyindole fumarate | | | | | |
| 1-(2-aminopropyl) 6- | 300 | 32.5 | 22.5 | 33.1 | 27.5 |
| methoxyindole fumarate | | | | | |
| 1-(2-aminopropyl) 5- | 300 | 39 | 1.2 | 16 | 20.1 |
| chloroindole fumarate | | | | | |

### METHOD 6

### Neuroprotective Effects in the Rat Photooxidative Induced Retinopathy Model

Male Sprague Dawley rats were randomly assigned to vehicle treatment (saline) or drug treatment (dissolved in saline) experimental groups. α-methyl-5-hydroxytryptamine (1, 5, and 10 mg/kg) or vehicle was administered by subcutaneous (SC) injection at 48, 24, and 0 hours prior to light exposure and once 24 hours after a 6-hour light exposure to spectrally filtered blue light (˜220 fc). Control rats were housed in their home cage under normal cyclic light exposure. Rats were single housed in clear polycarbonate cages during this light exposure.

The electroretinogram (ERG) was recorded after a five day recovery period from dark-adapted anesthetized rats (Ketamine-HCl, 75 mg/Kg; Xylazine, 6 mg/Kg). The eye's electrical response to a flash of light was elicited by viewing a ganzfeld. ERGs to a series of light flashes increasing in intensity were digitized to analyze temporal characteristics of the waveform and determine the response voltage-log intensity (VlogI) relationship. Changes in the ERG a-wave are associated with photoreceptor and retinal pigment epithelium damage while damage to the inner retina is reflected in changes in the ERG b-wave.

Rats dosed with α-methyl-5-hydroxytryptamine showed dose-dependent protection of outer and inner retina function against this photo-oxidative induced retinopathy (table 4). Maximum a- and b-wave response amplitudes in α-methyl-5-hydroxytryptamine (1.0 mg/kg) dosed rats were not significantly different than vehicle dosed rats and were approximately 30% of those of the control animals (Table 4). However, maximum a- and b-wave response amplitudes from α-methyl-5-hydroxytryptamine (5 and 10 mg/kg) dosed rats were approximately 58% and 62% of control animals. respectively, and significantly higher than responses measured in vehicle dosed rats (Table 4).

TABLE 4

| | | A-wave Amplitude | | B-wave Amplitude | |
|---|---|---|---|---|---|
| Treatment | N | Mean (μV) | SEM | Mean (μV) | SEM |
| Control | 6 | 829.8 | 41.9 | 1809.3 | 84.2 |
| Vehicle | 15 | 191.6 | 32.8 | 441.1 | 77.3 |

α-Methyl-5-hydroxytryptamine

| | | | | | |
|---|---|---|---|---|---|
| 1.0 mg/kg | 10 | 237.8 | 34.2 | 555 | 87 |
| 5.0 mg/kg | 9 | 471.93 | 48.9 | 1123 | 143.3 |
| 10.0 mg/kg | 10 | 477.7 | 51.2 | 1126 | 126.1 |

### METHOD 7

### Pharmacokinetic Studies in Rabbits

New Zealand Albino or Dutch-belted rabbits (3 to 5 per arm) can be dosed topically with a solution formulation of α-methyl-5-hydroxytryptamine (1%) in the right eye and with vehicle in the left eye twice a day for a period of up to one week. At the end of the dosing period the ocular fluids and tissues are collected and analyzed for the presence of the drug via HPLC analysis. The difference between the dosed eye and the contralateral vehicle dosed eye is a measure of the ability of the test item to penetrate directly to the retina/optic nerve head via topical ocular drug delivery. The drug concentrations in the vehicle dosed eye represent delivery from systemic circulation.

Compounds of this invention are dosed topically to the eye to lower and control IOP and treat glaucomatous optic neuropathy.

The Compounds can be incorporated into various types of ophthalmic formulations for delivery to the eye. The Compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving the Compounds in a physiologically acceptable isotonic aqueous buffer. Further, the ophthalmic solution may include an ophthalmologically acceptable surfactant to assist in dissolving the Compounds. Furthermore, the ophthalmic solution may contain an agent to increase viscosity, such as, hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the Compounds are combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-974, or the like, according to the published formulations for analogous ophthalmic preparations; preservatives and tonicity agents can be incorporated.

The Compounds are preferably formulated as a topical ophthalmic suspension or solution, with a pH of about 4 to 8. The Compounds will normally be contained in these formulations in an amount 0.003% to 5% by weight, but preferably in an amount of 0.01% to 2% by weight. Thus, for topical presentation 1 to 2 drops of these formulations would be delivered to the surface of the eye 1 to 4 times per day according to the discretion of a skilled clinician.

The compounds can also be used in combination with other agents for treating glaucoma, such as, but not limited to, β-blockers (e.g., timolol, betaxolol, levobetaxolol, carteolol, levobunolol, propranolol), carbonic anhydrase inhibitors (e.g., brinzolamide and dorzolamide), α₁ antagonists (e.g. nipradolol), α₂ agonists (e.g., iopidine and brimonidine), miotics (e.g., pilocarpine and epinephrine), prostaglandin analogues (e.g., latanoprost, travaprost, unoprostone, bimatoprost, and compounds set forth in U.S. Pat. Nos. 5,889,052; 5,296,504; 5,422,368; 5,688,819; and 5,151,444, “hypotensive lipids” (e.g., compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 06/203350, and appropriate compounds from WO94/13275, such as, memantine.

The following topical ophthalmaic formulations are useful according to the present invention administered 1-4 times per day according to the discretion of a skilled clinician.

### EXAMPLE 1

| Ingredients | Amount (wt %) |
|---|---|
| α-Methyl 5-hydroxytryptamine maleate | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 2

| Ingredients | Amount (wt %) |
|---|---|
| 1-(2-aminopropyl) 6-hydroxyindole | 0.01-2% |
| fumarate | |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 3

| Ingredients | Amount (wt %) |
|---|---|
| 1-(2-aminopropyl) 6-hydroxyindole | 0.01-2% |
| fumarate | |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### EXAMPLE 4

| Ingredients | Amount (wt %) |
|---|---|
| α-Methyl 5-hydroxytryptamine maleate | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

We claim:

1. A method for lowering and controlling IOP and treating glaucomatous optic neuropathy which comprises administering a pharmaceutically effective amount of a compound with both 5-HT₂ and 5-HT_{1A} agonist activity.

2. The method of claim 1 wherein the compound is selected from the group consisting of: α-methyl 5-hydroxytryptamine; α-methyl 5-methoxytryptamine; 1-(2-aminopropyl) 6-hydroxyindole; 1-(2-aminopropyl) 6-methoxyindole; and 1-(2-aminopropyl) 5-chloroindole.

3. A composition for lowering and controlling IOP and treating glaucomatous optic neuropathy comprising a pharmaceutically effective amount of a compound with both 5-HT₂ and ⁵-HT_{1A} agonist activity.

4. The composition of claim 3 wherein the compound is selected from the group consisting of: α-methyl 5-hydroxytryptamine; α-methyl 5-methoxytryptamine; 1-(2-aminopropyl) 6-hydroxyindole; 1-(2-aminopropyl) 6-methoxyindole; and 1-(2-aminopropyl) 5-chloroindole.

5. The use of a compound with both 5-HT₂ and 5-HT_{1A} agonist activity for the manufacture of a medicament useful for lowering and controlling IOP and treating glaucomatous optic neuropathy.

6. The use of claim 5 wherein the compound is selected from the group consisting of: α-methyl 5-hydroxytryptamine; α-methyl 5-methoxytryptamine; 1-(2-aminopropyl) 6-hydroxyindole; 1-(2-aminopropyl) 6-methoxyindole; and 1-(2-aminopropyl) 5-chloroindole.

7. The method of claim 1 which additionally comprises administering an additional agent.

8. The method of claim 7 wherein the additional agent is selected from the group consisting of: beta-blockers, carbonic anhydrase inhibitors, α₁ antagonists, α₂ agonists, miotics, prostaglandin analogues, hypotensive lipids, and neuroprotectants.

9. The method of claim 8 wherein the additional agent is a prostaglandin analogue.

10. The method of claim 9 wherein the prostaglandin analogue is selected from the group consisting of: latanoprost, travaprost, unoprostone, and bimatoprost.

11. The use of claim 5 which additionally comprises an additional agent.

12. The use of claim 11 wherein the additional agent is selected from the group consisting of: beta-blockers, carbonic anhydrase inhibitors, α₁ antagonists, α₂ agonists, miotics, prostaglandin analogues, hypotensive lipids, and neuroprotectants.

13. The use of claim 12 wherein the additional agent is a prostaglandin analogue.

14. The use of claim 13 wherein the prostaglandin analogue is selected from the group consisting of: latanoprost, travaprost, unoprostone, and bimatoprost.
